(19) United States
(12) Patent Application Publication
(10) Pub. No.: US 2024/0189346 A1
(43) Pub. Date: Jun. 13, 2024

(54) METHODS FOR RAISING NITRIC OXIDE AND REDUCTION OF URIC ACID

(71) Applicant: AZULENT, LLC, Las Vegas, NV (US)

(72) Inventors: Eugene P. PITTZ, Las Vegas, NV (US); Chen CHEN, Las Vegas, NV (US)

(21) Appl. No.: 18/583,657

(22) Filed: Feb. 21, 2024

Related U.S. Application Data
(63) Continuation of application No. PCT/US2023/011005, filed on Jan. 18, 2023.
(60) Provisional application No. 63/361,716, filed on Jan. 18, 2022.

(51) Int. Cl. A61K 33/06; A61K 9/00; A61K 31/522; A61K 33/00; A61K 45/06

(52) U.S. Cl. CPC A61K 33/06; A61K 9/0053; A61K 31/522; A61K 33/00; A61K 45/06

(57) ABSTRACT

A method of accelerating the production of nitric oxide (NO) can include co-administering a generator of H₂ with a generator of nitric oxide (NO) as a formulation to a subject. The generator of H₂ includes elemental magnesium metal powder and the generator of NO is a nitrate, while the formulation is also free of amino acids.

### CROSS REFERENCE TO RELATED APPLICATIONS

This application is a continuation of International Patent Application No. PCT/US2023/011005, filed Jan. 18, 2023, which claims priority to U.S. Provisional Patent Application No. 63/361,716, filed Jan. 18, 2022, each of which are incorporated herein by reference.

### INCORPORATION BY REFERENCE STATEMENT

Not applicable.

### BACKGROUND

Extensive research has shown the benefits of raising nitric oxide (NO) in the vascular system by providing: (1) substrate and/or modulator for endothelial nitric oxide synthase (eNOS); and (2) precursor and/or donor for the nitrate-nitrite-nitric oxide pathway. These benefits include reduction of hypertension, improving heart health, exercise performance and stamina. Thus, methods for increasing nitric oxide inside the body is of important therapeutic benefit.

On the other hand, elevated levels of serum uric acid, a product of the metabolism of xanthine by xanthine oxidase, has been strongly associated with bone and joint damage, gouty arthritis, kidney disease, heart disease, Alzheimer's disease, and vascular dementia. The lowering of high serum uric acid can be accomplished through diet and drugs such as allopurinol. However, dietary compliance is poor for most patients, and known effective drugs have toxic side effects. Thus, there is a need for a safe means of lowering uric acid that patients can readily incorporate into their daily diet.

### SUMMARY

Molecular hydrogen (H₂) in dosage forms of infused water and H₂ gas has been reported to raise systemic levels of H₂. However, up to this point, there is a lack of information as to how intrinsic production of H₂, by the microbiome, combined with generation and sustaining of H₂ by ingestion of magnesium metal powder, can affect the generation of both systemic molecular hydrogen and nitric oxide (NO).

It has been discovered that administering molecular hydrogen (H₂) can affect a reduction in uric acid and that combining delivery of H₂ with a specific dose level of ascorbic acid can add to the lowering of uric acid.

In some aspects, the techniques described herein relate to a method of accelerating the production of nitric oxide (NO) by co-administering a generator of H₂ with a generator of nitric oxide (NO) as a formulation to a subject, wherein the generator of H₂ includes elemental magnesium metal powder and the generator of NO is a nitrate, and the formulation is free of amino acids.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 is a chart of salivary nitric oxide as a function of time in accordance with one example.

FIG. 2 is a chart of hydrogen breath testing as a function of time in accordance with another example.

FIG. 3 is a chart of salivary nitric oxide from NEO40 as a function of time in accordance with one example.

FIG. 4 is a chart of breath hydrogen from NEO40 as a function of time in accordance with another example.

FIG. 5 is a chart of breath hydrogen as a function of time in accordance with an example of the present disclosure.

FIG. 6 is a chart of salivary uric acid as a function of time in accordance with an example of the present disclosure.

FIG. 7 is a chart of breath hydrogen as a function of time in accordance with an example of the present disclosure including comparative data.

FIG. 8 is a chart of salivary nitric oxide as a function of time in accordance with an example of the present disclosure, including comparative data.

FIG. 9 is a chart of salivary uric oxide as a function of time in accordance with an example of the present disclosure, including comparative data.

FIG. 10 is a chart of breath hydrogen as a function of time in accordance with an example of the present disclosure.

FIG. 11 is a chart of salivary nitric oxide as a function of time in accordance with an example of the present disclosure.

FIG. 12 is a chart of salivary uric acid as a function of time in accordance with several examples of the present disclosure.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a generator” includes reference to one or more of such materials and reference to “the enzyme” refers to one or more of such biological catalysts.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of” For example, “at least one of A, B and C” explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Example Embodiments

A method of accelerating the production of nitric oxide (NO) can include co-administering a generator of H₂ with a generator of nitric oxide (NO) as a formulation to a subject. These formulations can result in intrinsic and extrinsic production of molecular hydrogen to increase systemic nitric oxide and lower levels of uric acid for therapeutic purposes.

The generator of H₂ includes elemental magnesium metal powder. Elemental magnesium powder is magnesium metal having an oxidation state of zero. Elemental magnesium powder is distinct from ionic magnesium (e.g. provided as a metal salt or as a chelate) and magnesium compounds (e.g. magnesium oxide, etc.). Elemental magnesium powder can react with water to form hydrogen and magnesium hydroxide. Hydrogen is readily absorbed from the GI tract into systemic circulation (see Examples below). Introduction of H₂ in the upper GI tract along with production of the antacid magnesium hydroxide can reduce the symptoms of gastritis including pain and inflammation.

The magnesium metal powder can be provided as a granular or particulate form which typically ranges in size from about 0.1 μm to about 100 μm, although sizes up to about 500 μm may be useful. As a general guideline, the magnesium metal powder is in the formula at a concentration of 0.01 to 50% w/w, and in some cases 0.02 to 15% w/w when in a dry form. When the formulation is provided mixed with water, or in an aqueous gel, the magnesium metal powder can be 0.001 to 0.50% w/v.

Any base metal powder, at the same concentration range could be used alone or in combination for generating H₂ including manganese, zinc, calcium, etc.

In some cases, the generator of H₂ further includes hydrogen-infused water. Hydrogen-infused water can impart additional anti-inflammatory, anti-oxidant, anti-anxiety, and other benefits to the formulation. Hydrogen infused water can also be generated by electrolysis.

In other cases, the generator of H₂ further includes H₂ gas which is administered by inhalation. Such H₂ gas can be produced by electrolysis, dissolving a tablet, direct dispensing from a pressurized container, or the like. Non-limiting examples of H₂ gas sources for inhalation include large electrolysis machines.

In another alternative, the generator of H₂ further includes ascorbic acid. The ascorbic acid can generally be present in the formulation at about 0.001 to 75% w/w, and in some cases 0.01 to 15%. The use of ascorbic acid can also increase salivary nitric oxide (SNO) test strip measurements by up to 350%. Notably, the enhanced effect of ascorbic acid is pH dependent. As a general guideline, ascorbic acid can be present at an acidic pH, and in some cases a pH below 4.0. Similarly, increasing pH will slow production of hydrogen. Thus, the pH can be adjusted to either accelerate or delay production of hydrogen.

In the formulations, the generator of nitric oxide is a nitrate. Without being bound to a specific theory, it appears that the in vivo mechanism by which nitrate is converted to nitric oxide via nitrate-nitrite-nitric oxide pathway following the entero-salivary circulation of inorganic nitrate. The nitrate-nitrite-nitric oxide pathway is complementary to the classical eNOS pathway in which a guanidine nitrogen on arginine is oxidized to generate citrulline and also NO as a byproduct. This alternative pathway normally provides about half of nitric oxide in the vascular system and becomes more important when the eNOS pathway is impaired such as seen with aging. Briefly, inorganic nitrate such as dietary nitrate in green leafy vegetables is ingested and absorbed into the circulation. Circulating nitrate including the nitrate formed from oxidation of endogenously produced NO from the NOS enzymes is transported into the saliva gland and concentrated in the saliva. If secreted in the mouth, nitrate in the saliva is reduced to nitrite by nitrate reducing bacteria. Once swallowed, part of the nitrite is chemically reduced to NO under the acidic condition in the stomach and the rest can be reduced to NO by several different enzymes such as xanthin oxidase after being absorbed. The two nitric oxide generation pathways are interconnected inside the body and nitrate serve as a storage pool of nitric oxide. The reduction of nitrate to nitrite by nitrate reducing bacteria in the mouth is obligatory for the generation of nitric oxide from nitrate. As such, salivary nitrite is routinely measured by nitric oxide saliva test strips as a surrogate for systemic NO.

In one example, the generator (or a precursor, a donor) of NO is present at a concentration of 0.01-50% w/w, and in some cases 0.1 to 5.0% w/w. Non-limiting examples of suitable generators of NO include a salt of nitrate, a salt of nitrite, beet juice powder, celery powder, or any vegetable powder containing nitrates. As non-limiting examples, in a powder formulation the nitrate can be in a salt form, including one or more of sodium, potassium, magnesium, calcium, zinc, ammonium, aluminum, barium, bismuth, copper, iron, lithium, manganese, silver, and thiamine. Most often, the nitrate is provided from a natural product.

In another example, the nitrate is provided in an herbal powder. Non-limiting examples of nitrate herbal powders can include green coffee bean, danshen root (Radix saliva mitorrhizae), snake gourd fruit (Fructus trichosanthis), longstamen onion bulb (Bulbus allii macrostemi), sanchi (Radix notoginseng, ginseng (Radix ginseng), borneol (Borneolum syntheticum), and borneol (Cinnamomum, or combinations of such). In one example, the natural product is a vegetable powder. Non-limiting examples, of vegetable powder can include arugula, beet, cabbage, celery, cress, fennel, leek, spinach, lettuce, mustard green, parsley, swiss chard, leafy chicory, kohlrabi, radish, or a combination of such. In still another example, the natural product is a fruit powder. Non-limiting examples, of fruit powder can include watermelon, apple, banana, grape, Kiwi, nectarine, peach, pomegranate, pears, oranges, or combinations of such.

The formulation can also be free of amino acids. Amino acids can negatively affect pH of the formulation. Although pH can vary, the formula can have an acidic pH, and in some cases less than about 5, and in other cases less than about 4.

In yet another aspect, the method can include ingesting a high fiber diet, which generates molecular hydrogen, before the administering the generator of NO. As an example, the high fiber diet can contain one or more of the following fibers at a total fiber content of 3-60 grams/serving: beans, legumes, whole grains, cereals, vegetables, fruits, nuts, seeds, cellulose, hemicellulose, psyllium, resistant starch, inulin, wheat dextrin, oligosaccharides, pectin, glucomannan, xanthan gum, carrageenan, and combinations thereof.

Although a delay in ingesting the generator of NO after the high fiber diet can vary considerably, as a general guideline, the high fiber diet can be ingested 2-8 hours before ingestion of the generator of NO. NO will be generated from nitrite in the upper GI tract as well as after intestinal absorption of nitrite while microbiome generated H₂ takes place in the lower GI tract, accounting for the difference in time of NO generation and H₂ generation.

In another example, the formulation can further comprise a modulator of endothelial nitric oxide synthase (eNOS). A modulator includes an activator, cofactor, and any other agents that increase eNOS expression and activity. Generally, the modulator of eNOS can be present in the formulation up to 25% w/w.

Non-limiting examples of suitable activator and cofactors of eNOS isoenzymes includes at least one of nitroglycerin, acetylsalicylic acid, BH₄ (tcetrahydrobiopterin), folic acid, vitamin B₁, vitamin B₆, vitamin B₁₂, vitamin D₃, magnesium, and zinc. As a general guideline, the modulator of eNOS can be present in the formulation in an effective amount which may vary depending on the choice of activator. However, as a general guideline, the eNOS activator can be present in the formulation at 0.01 to 50% w/w.

In another alternative, the formulation can further comprise one or more of uric acid control agents such as xanthine oxidase inhibitors (e.g. allopurinol), anti-inflammatory steroids such as corticosteroids, and colchicine. These drugs can reduce uric acid but tend to also have negative side effects that can be reduced by combining with H₂. This benefit can occur either through the anti-inflammatory effects of H₂ and/or by reduction of the dosage for the drug to be combined with H₂. Natural compounds to lower uric acid include polyphenols (i.e., quercetin), organosulfur compounds (i.e., S-allyl cysteine), etc. Accordingly, introduction of a uric control agent can mitigate negative side effects from the above drugs and other similar drugs. Although percentages may vary, such uric acid control agents can be present in the formulation at 0.01 to 50% w/w.

The formulation can be provided as a dry product such as in the form of capsules, tablets, powders, or hydrogels. In another example, the formulation can be provided as a liquid. Thus, additional optional ingredients in the formulation can include, but are in no way limited to, preservatives, chelating agents, bulking agents, fillers, carriers, binders (e.g. corn starch, gelatin, celluloses, sucrose, lactose, etc.), disintegrants (e.g. crosslinked cellulose, modified starch, etc.), anti-adherents (e.g. magnesium stearate, calcium stearate, for example), colorants (e.g. titanium dioxide, carmine, for example), glidants (e.g. fumed silica, talc, magnesium carbonate, for example), lubricants or anti-caking agents (e.g. talc, silicon dioxide, magnesium stearate, calcium stearate, stearic acid, for example) preservatives, desiccants, coatings (e.g. for tablets), flavorants, pH adjusters, sweeteners, other excipients, and the like. In the case of a capsule, the capsule (e.g. container) can be formed of materials such as hydroxypropyl methylcellulose, gelatin, carboxymethyl cellulose, hypromellose, and the like. Aqueous gels, including those formed due to glucomannan, xanthan gum, chitosan oligosaccharides, combinations of such and any biopolymers can be present at a concentration of 0.2-5% w/v of the hydrogel.

Administration and ingestion of the formulation can be sufficient to result in certain therapeutic effects. For example, the administering of the generator of H₂ can be sufficient to lower a blood level of uric acid. The blood level of uric acid can be determined by measuring a salivary uric acid level. In some cases, the administering of the generator of H₂ is sufficient to reduce pain due to inflammation affected by uric acid. Ascorbic acid, for example, in an amount up to about 50% w/w can further reduce uric acid. The amount sufficient to achieve these therapeutic effects can vary by individual, severity of symptoms and other factors. However, as a general guideline, for reduction of uric acid, 250 mg/day is an effective lower limit while an upper limit will depend on the specific generator of H₂ but should be below toxicity levels for the particular generator. Thus, amounts within this range can be targeted. Further, combining H₂ with ascorbic acid can be expected to lower the dose of ascorbic acid effective in achieving lower levels of uric acid.

These formulations can be of particular benefits when specifically targeting individuals having certain diseases which involve NO deficiencies. In some cases, the subject is identified prior to the co-administering as having one or more diseases including cardiovascular disease, bone and joint deterioration, gouty arthritis, kidney disease, Alzheimer's disease, and vascular dementia. The above formulations can then be administered and SNO levels monitored subsequent to ingestion of the formulations. This monitoring can occur at intervals of 30 minutes, 1 hour, 6 hours, or up to several days subsequent to ingestion. Generally, SNO variations can be measured at times of at least about 15 minutes subsequent to ingestion.

### Example 1: Interaction of Molecular Hydrogen (H₂) and Potassium Nitrate (KNOs) in Increasing Salivary Nitric Oxide

This Study was conducted to investigate the effect of a powder formulation containing 400 mg of potassium nitrate (KNO₃) and 70 mg of magnesium metal powder (MMP), plus excipients, on the time course of salivary nitric oxide (NO) as compared to a control formulation without magnesium metal powder (MMP).

### Experimental

The powder base for all three formulations consisted of the following excipients: anhydrous citric acid, glucomannan, xanthan gum, L-arginine, L-citrulline maleate, sodium ascorbate, vitamin E powder, riboflavin, and stevia. In addition, the formulas evaluated are as follows: MMP: 70 mg of 200-325 Mesh magnesium metal powder in 6.86 grams of powder formula; KNO₃: 400 mg of potassium nitrate in 6.86 grams of powder formula; MMP/KNO₃: 70 mg of 200-325 Mesh magnesium metal powder plus 400 mg of potassium nitrate in 6.86 grams of powder formula.

These formulas were each mixed with 100 mL of reverse osmosis water just before dosing. Salivary NO was determined using Berkeley Test® Nitric Oxide (NO) Saliva Test Strips which detect nitrite as a surrogate for nitric oxide. Molecular hydrogen (H₂) was measured with an H₂ Forensics Detector® modified for breath testing. The three studies were conducted on three separate days under the same conditions of overnight fasting, not eating during the Studies and starting the Studies early in the morning.

### Results

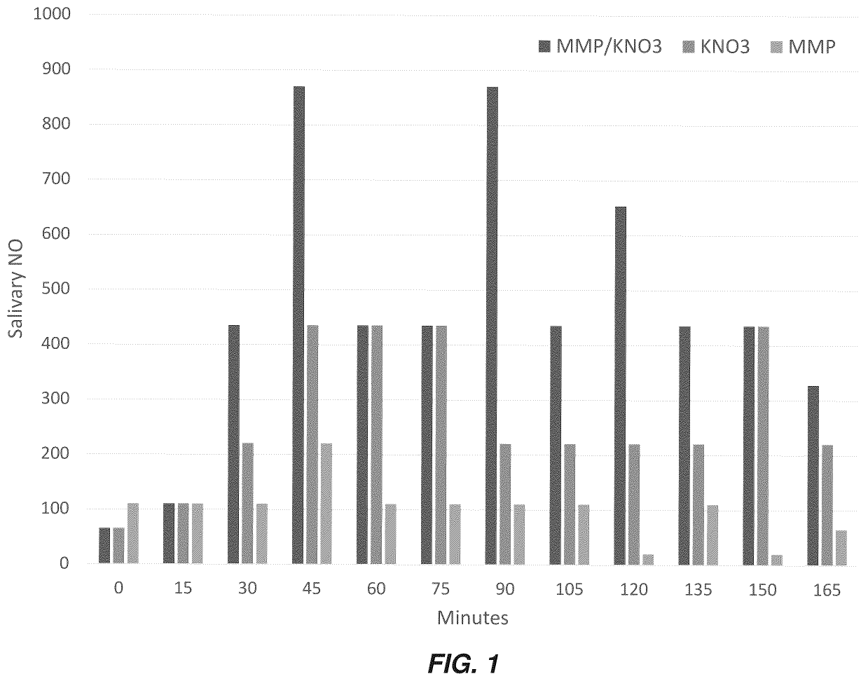

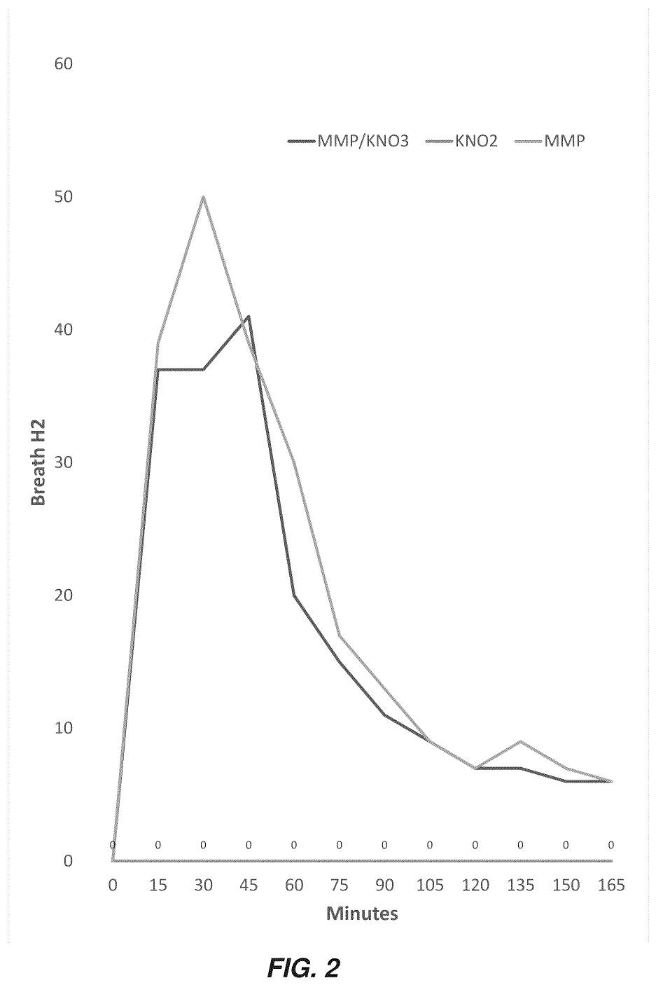

The results are displayed in FIGS. 1 and 2.

The bar graph of FIG. 1 displays the time course of salivary NO testing. It can be observed that the test formulation containing both 400 mg KNO₃ and 70 mg MMP affects higher levels of salivary NO at 7 of 11 time-dependent measurements. The formulation containing 400 mg KNO₃ and 70 mg MMP affects a higher level of salivary NO as early as 30 minutes after ingestion. There was no time point measurement when the formulations containing just KNO₃ or just MMP (controls) affected a higher level of salivary NO compared to the combination of MMP/KNO₃ dosing. FIG. 2 displays the time course of breath H₂.

### Discussion

The results presented here support the assertion that H₂ generated from dosing with MMP, can have both an effect of reducing the time for generating salivary NO and an enhanced production of NO from KNO₃.

### Example 2: Nitric Oxide Production Due to Administration of NEO40® with and without the Generation of Molecular Hydrogen (H₂)

The presence of intestinal H₂ gas (determined by breath H₂) increases salivary nitric oxide (SNO) due to dosing with a commercial product containing nitrite and nitrate (NEO 40®). Co-administration of NEO40 with a generator of up to 28 ppm H₂ (H₂-Boost™ capsules) not only markedly increases SNO, but reduces the time course for generation of NO.

Although molecular hydrogen, in the dosage forms of H₂-infused water and H₂ gas can increase nitric oxide levels, there is a need to study the time course of the effect of H₂ on the levels of NO. In the process of doing so, it has been discovered that H₂ reduces the time course of generation of NO and intestinal H₂ gas, generated from fibrous food increases NO.

### Experimental

Three studies were conducted on three consecutive days, under similar conditions, when 8-9-hours of fasting had taken place overnight. As instructed, a single tablet of NEO 40® was taken at the start of each study, along with 14-oz of water. NEO40® contains generators of NO, including beet root powder, Hathorne berry extract, L-citrulline and sodium nitrite.

Salivary nitric oxide (SNO) was determined with Berkeley Nitric Oxide Saliva Test Strips while breath H₂ was measured with the Forensics Detectors instrument. For one Study (Zero H₂) no breath H₂ was detectable during the study. For the second Study (Nat. H₂), a modest level of breath H₂ was available through the Study. This modest ‘natural’ level of H₂ was due to the ingestion of a large bowel of black beans on the previous evening around 6:00 PM. For the third study (Ingest H₂), two capsules of H₂-Boost™ were taken along with a tablet of Neo40® and 14 oz of water. An H₂-Boost capsule contains 80 mg of magnesium metal powder and excipients. One capsule was opened, and its contents were mixed with the drinking water while the other capsule was consumed intact.

### Results

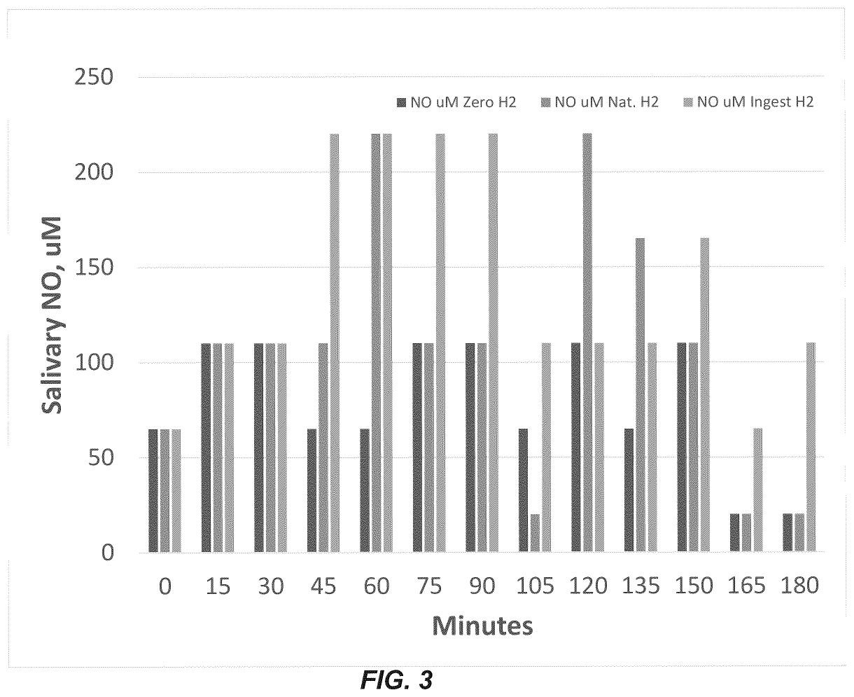

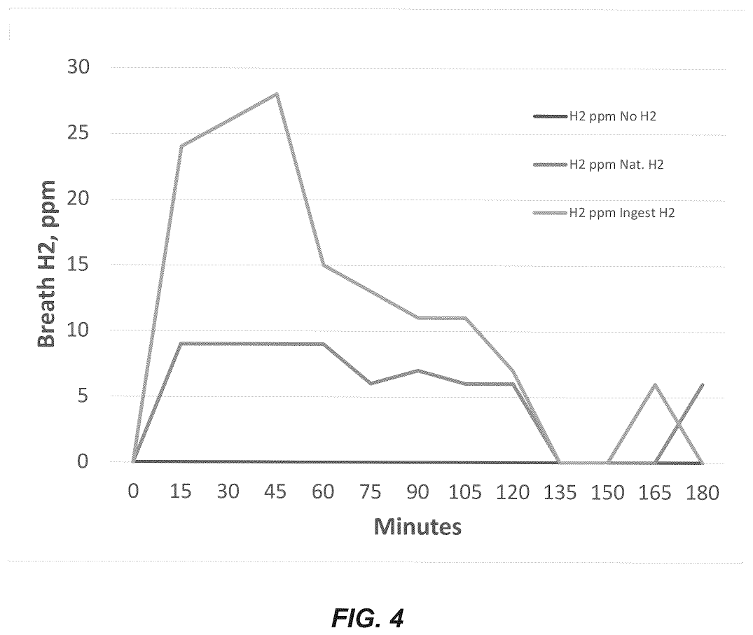

The results are presented in FIGS. 3 and 4. NE040®, manufactured by HumanN®, is labeled to be a nitric oxide producing formula that provides ‘Daily Heart & Circulation Support’. The list of ingredients shows that it contains vitamin C, vitamin B₁₂, a proprietary nitric oxide blend (beet root powder, Hawthorne berry extract, L-citrulline and sodium nitrite) and excipients.

During the first Study, as shown in FIG. 3, salivary NO (SNO) testing of NEO40 administration, without any detectable breath H₂ (Zero H₂— blue line of FIG. 4) either did not raise SNO above a low baseline level or raised it from a depleted level (20 μM) to a low level (100 μM). The results are shown in FIG. 4. This time course Study was conducted early in morning when the subject has had a modest level of intestinal gas most likely due to his eating a bowel of black beans the previous evening.

During the second Study, a modest increase, above baseline, in breath H₂ occurs through most time point measurements. By comparing the blue with the red line of the bar graph of FIG. 3, it can be seen that on 4 of 12 of the time point measurements, the presence of intestinal H₂ gas (Nat. H₂), as indicated by breath H₂, raised SNO. In contrast, only on one occasion (105 minutes), was there a higher level of SNO when there was an absence of H₂. Of more significance is the observation that in the presence of Nat H₂, SNO reached threshold target (150-220 μM) values on three occasions.

For the third Study (grey line), an up to 28 ppm level of H₂ was present through the time course due to co-administration of H₂-Boost™ capsules and NEO40® as described above. As a result, and as shown in the bar graph, on five of twelve occasions SNO has reached the threshold value (150-220 μM) which would be considered an improvement in SNO due to use of a product.

For the third Study, there appears to be a phase lag where breath H₂ rises from 0 to 45 minutes post-dosing while SNO does not rise above the ‘low’ level until 45 minutes post-dosing. The reason for the phase lag is unknown, but it can be speculated that H₂ may be shifting the metabolism of nitrite away from peroxynitrite toward NO.

### Example 3: Effect of Molecular Hydrogen on High Uric Acid

Chronic, elevated levels of uric acid (UA) are associated with metabolic diseases including cardiovascular disease and gouty arthritis. Here, it is demonstrated that oral administration of an H₂ generating protocol lowers SUA (salivary uric acid) from a chronically elevated level of 750 micromoles (μM). There is a phase lag where after the administration of H₂, sixty-minutes passes before SUA becomes lowered. SUA remains lower than the baseline SUA for 3-4-hours when it again returns to baseline levels. The mechanism by which H₂ lowers UA is unknown but may be due to its potential to reduce toxic reactive oxygen species (ROS) that are generated when purines are metabolized to UA. That is, H₂ may interfere with the synthesis of UA.

Chronically high UA could be due to a high rate of synthesis of UA, a slow elimination of UA by the kidneys or both. High UA can be treated with drugs, such as allopurinol, which are effective at inhibiting the synthesis of UA, but can actually make the situation worse by affecting kidney damage and kidney failure.

UA can function as both a pro-oxidant and as an antioxidant. It appears that UA acts as an antioxidant when in the systemic circulation, but UA acts as a pro-oxidant inside of cells. The metabolism of purines to UA, itself, affects the production of ROS. Regardless, lowering salivary UA from chronically elevated levels, i.e., 750 μM UA, to levels considered to be free of increasing metabolic disease risk below 325 μM UA is desirable. SUA has been shown to be correlated with plasma UA.

### Experimental

H₂-Boost™ capsules were used. Each capsule contains 80 mg of MMP and excipients, as shown in Example 2. One 000 HPMC capsule, containing 1.08 grams of formulation was opened, and its contents mixed with 14-oz of water. Another capsule was left intact and ingested along with the water. Breath H₂ was measured with a modified Forensics Detectors® instrument. Salivary uric acid (SUA) was determined with Berkeley Uric Acid Test Strips, as instructed.

### Results

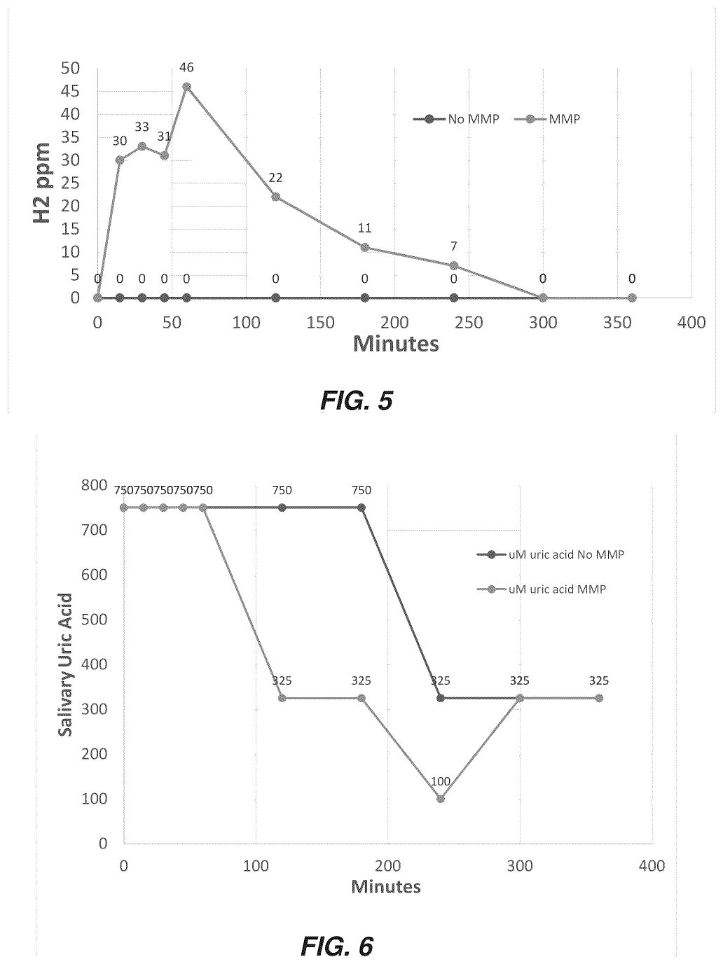

The results are shown in FIGS. 5 and 6. The results indicate that administration of an H₂-generating product lowers SUA. Since SUA is correlated with serum UA, H₂ administration can be assumed to also lower serum UA. FIG. 6 demonstrates that H₂ (MMP as a generator) reduces the time for lowering SUA, by 50% by about 115 minutes. The mechanism by which H₂ lowers UA can be speculated upon. For example, H₂ may alter the metabolic pathway that generates UA. The metabolism of purines through xanthine to uric acid involves the generation of ROS. If H₂ squelches these ROS, it could be a means by which H₂ reduces UA.

Regardless of the mechanism, this example includes the discovery that an approximately one-hour phase lag between the appearance of breath H₂ and the drop in UA and supports a mechanism by which H₂ acts to lower UA. In this experiment, the drop in UA appears to last for about 4 hours. The increased H₂ level lasts for 4-5 hours.

### Example 4: Effect of Molecular Hydrogen and Nitric Oxide on Salivary Uric Acid

Studies were conducted to determine the combined effect of oral delivery of molecular hydrogen and NaNO₃ (i.e., a generator of NO) on salivary uric acid.

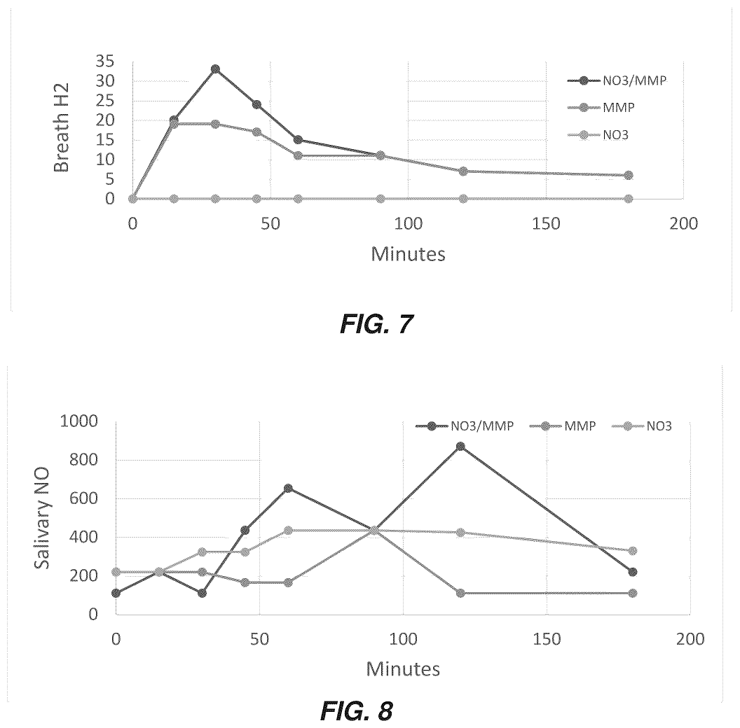

The studies were conducted on three consecutive days when the subject had fasted overnight, about 8-hours, and did not consume food during the Studies. The time course studies a shown in FIGS. 7, 8, and 9. The 000 HPMC capsular formulations studied contained:
Red Line: Magnesium metal powder (80 mg) plus excipients*.
Blue Line: Magnesium metal powder (80 mg) plus sodium nitrate (450 mg) plus excipients*.
Grey Line: Sodium nitrate (450 mg) plus excipients*.
Each capsule contained 1.13 grams of Formulation *Excipients are dextrose (DEX), glucomannan and xanthan gum.

The test procedures are as follows: Baseline measurements were taken before dosing. The contents of a designated 000 HPMC capsule were added to 7-ounces of water and briefly mixed, then consumed, followed by consuming another 7-ounce glass of water. Measurements of breath H₂, salivary NO and salivary uric acid at the time-points indicated in FIGS. 7, 8, and 9.

The results plotted in FIG. 7 indicate that the presence of NaNO₃ in the formulation with magnesium metal powder may have an effect of increasing output of H₂ within 30 minutes of dosing.

The results plotted in FIG. 8 demonstrate that the presence of magnesium metal powder along with sodium nitrate affect an increase in NO—at 60 and 120 minutes, post dosing relative to the formulation containing sodium nitrate without magnesium metal powder.

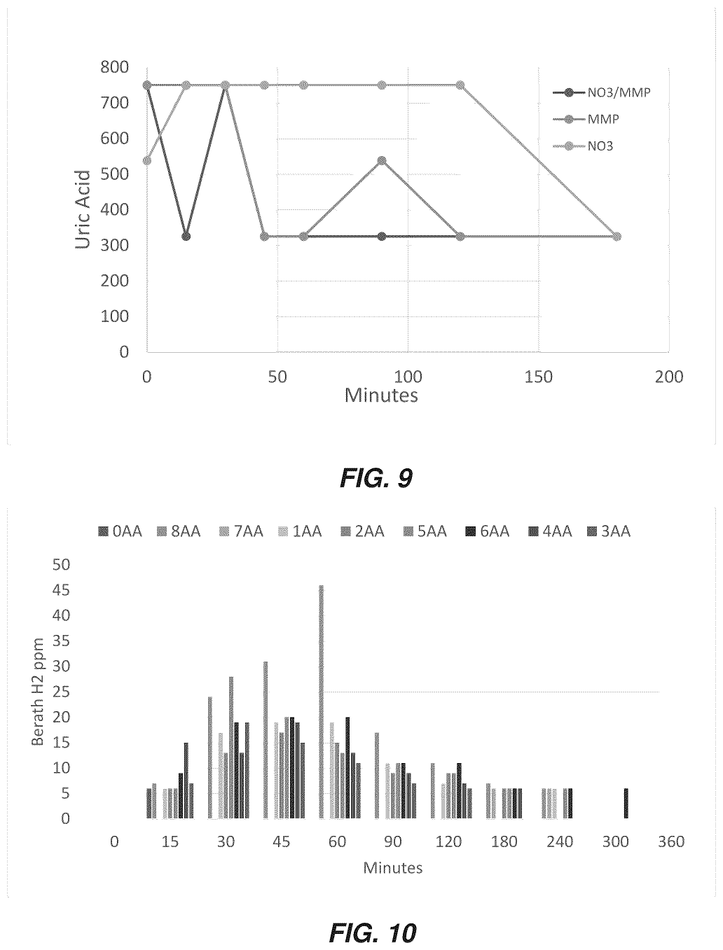

The results plotted in FIG. 9 show that the formulation containing sodium nitrate either sustains or raises salivary uric acid for 120 minutes post-dosing. Both formulations, containing molecular hydrogen, lower uric acid relative to the formulation containing sodium nitrate without molecular hydrogen. Thus, these Studies provide additional supporting evidence that molecular hydrogen can lower salivary uric acid.

### Example 5: Effect of Ascorbic Acid, Molecular Hydrogen and Nitric Oxide on Salivary Uric Acid—when Consuming Formulations Containing Ascorbic Acid, Magnesium Metal Powder and Sodium Nitrite

This Study was conducted to understand the effect on uric acid of molecular hydrogen (H₂) and nitric oxide (NO) in the presence and absence of ascorbic acid. The agents of interest, along with the excipients, konjac glucomannan and dextrose were orally delivered from HPMC 000 capsules.

Breath H₂, in ppm, was measured with a modified Forensics Detectors® instrument, salivary nitric oxide (NO) and salivary uric acid, both in μM units, were estimated using Berkeley test strips.

### Results

The results are depicted in Tables 1-3 and FIGS. 10-12.

TABLE 1
Breath H₂ as a function of time for several different formulas.

| H2, ppm | 0AA | 8AA | 7AA | 1AA | 2AA | 5AA | 6AA | 4AA | 3AA |
|---|---|---|---|---|---|---|---|---|---|
| Minutes | Control | No NaNO₃ | No MMP | No AA | 0.05 AA | 0.40 AA | 0.8 AA | 0.2 AA | 0.1 |
| 0 | 0 | 0 | 0 | 0 | 0 | 0 | 0 | 0 | 0 |
| 15 | 6 | 7 | 0 | 6 | 6 | 6 | 9 | 15 | 7 |
| 30 | 0 | 24 | 0 | 17 | 13 | 28 | 19 | 13 | 19 |
| 45 | 0 | 31 | 0 | 19 | 17 | 20 | 20 | 19 | 15 |
| 60 | 0 | 46 | 0 | 19 | 15 | 13 | 20 | 13 | 11 |
| 90 | 0 | 17 | 0 | 11 | 9 | 11 | 11 | 9 | 7 |
| 120 | 0 | 11 | 0 | 7 | 9 | 9 | 11 | 7 | 6 |
| 180 | 0 | 7 | 6 | 0 | 6 | 6 | 6 | 6 | 0 |
| 240 | 0 | 6 | 6 | 6 | 0 | 6 | 6 | 0 | 0 |
| 300 | 0 | 0 | 0 | 0 | 0 | 0 | 6 | 0 | 0 |
| 360 | 0 | 0 | 0 | 0 | 0 | 0 | 0 | 0 | 0 |
| 420 | 0 | 0 | 0 | 0 | 0 | 0 | 0 | 0 | 0 |
| Sum | 6 | 149 | 12 | 85 | 75 | 99 | 108 | 82 | 65 |

* All formulations contain 0.13 g glucommannan (GMN), no food with 1 intact capsule
0AA: Nothing—baseline with no formulation dosage.
8AA: GMN/0.13 g/NaNO₃ 0.0 g/DEX 0.58 g/AA 0.80 g/MMP 0.08 g
7AA: GMN 0.13 g/NaNO₃ 0.5 g/DEX 0.16 g/AA 0.80 g/MMP 0.0 g
1AA: GMN 0.13 g/NaNO₃ 0.5 g/DEX 0.88 g/AA 0.0 g/MMP 0.08 g
2AA: GMN 0.13 g/NaNO₃ 0.5 g/DEX 0.83 g/AA 0.05 g/MMP 0.08 g
5AA: GMN 0.13 g/NaNO₃ 0.5 g/DEX 0.4 g/AA 0.4 g/MMP 0.08 g
6AA: GMN 0.13 g/NaNO₃ 0.5 g/DEX 0.07 g/AA 0.80 g/MMP 0.08 g
4AA: GMN 0.13 g/NaNO₃ 0.5 g/DEX 0.67 g/AA 0.20 g/MMP 0.08 g
3AA: GMN 0.13 g/NaNO₃ 0.5 g/DEX 0.73 g/AA 0.1 g/MMP 0.08 g

From Table 1 and FIG. 10, it can be observed that a formulation (8AA), devoid of NaNO₃, generates a much higher H₂ (45 ppm) peak level and higher H₂ in AUC units (Area Under the Curve, i.e., Sum=149 ppm/420 min) than does formulations containing NaNO₃, along with excipients, or the controls (0AA, 7AA) without MMP.

From Table 1 and FIG. 2, it is observed that a formulation (6AA) containing the highest dose of ascorbic acid (0.80 g) sustains H₀ for 1-hour (25%) longer. This effect is not observed at lower doses of ascorbic acid.

From Table 1 and FIG. 2, it is observed that a formulation (7AA) devoid of magnesium metal powder (MMP) but containing 0.80 grams of ascorbic acid raised H₂ to 6 ppm at the 180- and 240-time measurements relative to 0AA, the control without ascorbic acid or magnesium metal powder.

TABLE 2
Effect on salivary NO over time for various formulations.

| μM | 0AA | 8AA | 7AA | 1AA | 2AA | 5AA | 6AA | 4AA | 3AA |
|---|---|---|---|---|---|---|---|---|---|
| Minutes | Control | No NaNO₃ | No MMP | No AA | 0.05 AA | 0.40 AA | 0.8 AA | 0.2 AA | 0.1 AA |
| 0 | 65 | 165 | 110 | 65 | 220 | 110 | 110 | 110 | 220 |
| 15 | 110 | 220 | 110 | 65 | 110 | 20 | 328 | 110 | 220 |
| 30 | 110 | 220 | 220 | 435 | 110 | 65 | 328 | 165 | 870 |
| 45 | 20 | 110 | 220 | 870 | 435 | 328 | 653 | 435 | 870 |
| 60 | 65 | 220 | 220 | 435 | 435 | 328 | 165 | 653 | 870 |
| 90 | 20 | 110 | 328 | 220 | 435 | 435 | 435 | 435 | 653 |
| 120 | 65 | 220 | 653 | 435 | 220 | 328 | 435 | 328 | 435 |
| 180 | 65 | 165 | 435 | 435 | 165 | 220 | 435 | 435 | 328 |
| 240 | 20 | 220 | 220 | 328 | 328 | 328 | 328 | 165 | 328 |
| 300 | 20 | 220 | 435 | 110 | 110 | 165 | 220 | 220 | 328 |
| 360 | 20 | 110 | 110 | 110 | 435 | 65 | 435 | 65 | 110 |
| Sum | 515 | 1815 | 2951 | 3443 | 2783 | 2282 | 3762 | 3011 | 5012 |

* All formulations contain 0.13 g glucomannan (GMN), no food + 1 intact capsule.

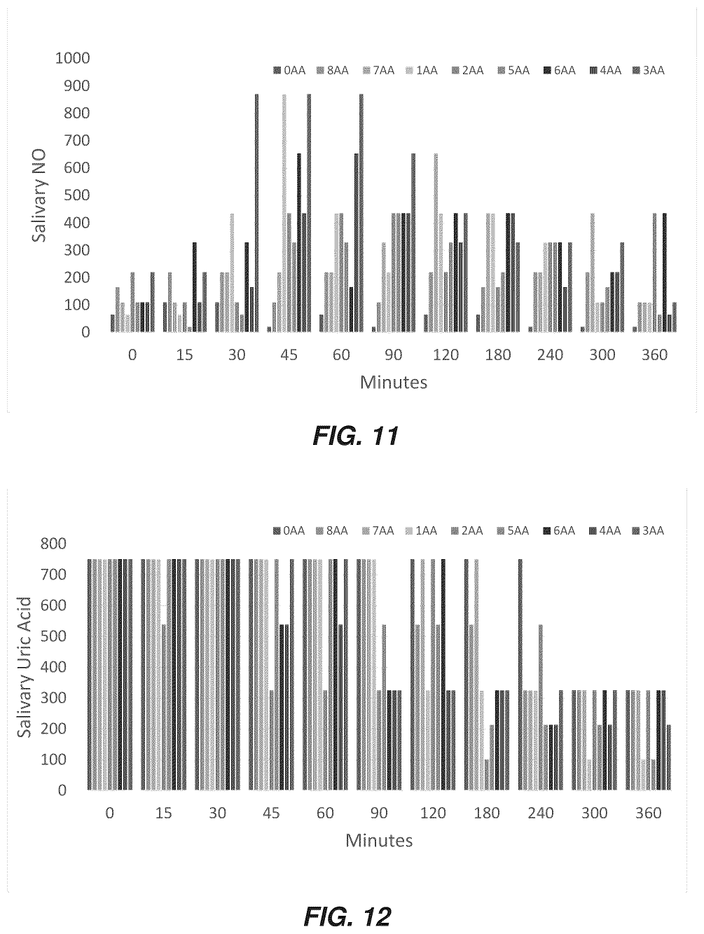

From Table 2 and FIG. 11, it can also be observed that for the formulation without ascorbic acid, (0AA), the AUC (515 μM/360 minutes) is much lower than the levels affected by all other formulations evaluated.

Remarkably, as can be observed in Table 2 and FIG. 11, a formulation that does not contain NaNO₃ but does contain MMP, as well as containing the highest dose of ascorbic acid (8AA) studied raises the total output of NO by 352% (Compare Columns 2 & 3 of Table 2). Thus, it has been discovered that a combination of molecular hydrogen and ascorbic acid can increase endogenous nitric oxide without dosing with a nitric oxide precursor and/or promoter. Thus, the combination raises endogenous nitric oxide.

From Table 2 and FIG. 11, it can be observed that a formula containing 0.1 g of ascorbic acid along with MMP and NaNO3 (3AA) delivers the highest peak NO as well as the most NO (AUC=5021 μM/360 min.). Thus, ascorbic acid affects a biphasic response on the generation of NO under these conditions.

TABLE 3
Effect of salivary uric acid over time for several formulations.

| μM | 0AA | 8AA | 7AA | 1AA | 2AA | 5AA | 6AA | 4AA | 3AA |
|---|---|---|---|---|---|---|---|---|---|
| Minutes | Control | No NaNO₃ | No MMP | No AA | 0.05 AA | 0.40 AA | 0.8 AA | 0.2 AA | 0.1 AA |
| 0 | 750 | 750 | 750 | 750 | 750 | 750 | 750 | 750 | 750 |
| 15 | 750 | 750 | 750 | 750 | 538 | 750 | 750 | 750 | 750 |
| 30 | 750 | 750 | 750 | 750 | 750 | 750 | 750 | 750 | 750 |
| 45 | 750 | 750 | 750 | 750 | 325 | 750 | 538 | 538 | 750 |
| 60 | 750 | 750 | 750 | 750 | 325 | 750 | 750 | 538 | 750 |
| 90 | 750 | 750 | 750 | 750 | 325 | 538 | 325 | 325 | 325 |
| 120 | 750 | 538 | 750 | 325 | 750 | 538 | 750 | 325 | 325 |
| 180 | 750 | 538 | 750 | 325 | 100 | 213 | 325 | 325 | 325 |
| 240 | 750 | 325 | 325 | 325 | 538 | 213 | 213 | 213 | 325 |
| 300 | 325 | 325 | 325 | 100 | 325 | 213 | 325 | 213 | 325 |
| 360 | 325 | 325 | 325 | 100 | 325 | 100 | 325 | 325 | 213 |
| Sum | 6650 | 5801 | 6225 | 4925 | 4301 | 4815 | 5051 | 4302 | 4838 |

* All formulations contain 0.13 g glucomannan (GMN), no food with 1 intact capsule

From observing Column 2 of Table 3 and FIG. 12 it can be seen that the baseline control (0AA) shows a sustained elevated level of salivary uric acid (SUA) of 750 μM up to 240 minutes. Thereafter, SUA falls to a more normal level of 325 μM.

From Table 3 and FIG. 12, it can be observed that all of the formulations studied, have an effect of lowering SUA. However, the formulation devoid of MMP (7AA)—has the least effect (i.e., AUC 6650 vs. 6225 μM for the 0AA control) as seen by comparing Column 2 and Column 4. This result demonstrates that ascorbic acid, without the presence of MMP (i.e., without H₂) has positive but weak effect on additional lowering salivary levels of UA as compared to formulations containing MMP.

From Table 3 and FIG. 12, it can be observed that the formulation with the lowest amount of ascorbic acid and containing MMP (2AA) has the fastest acting effect on lowering SUA where lowering SUA takes place as early as 45 minutes post-dosing. 2AA along with 4AA also have strongest effects at lowering the AUC of SUA (See Columns 6 and 9).

The results of these Studies point to interesting findings. For example, the results indicate that H₂ raises endogenous NO. This very interesting effect may be due to a shifting of nitrite metabolism to NO, rather than progressing to peroxynitrite. The results also indicate that H₂ lowers SUA. The presence of the lowest dose of ascorbic acid with MMP, seems to lower SUA at the earliest point. The presence of NaNO₃ may be a contributing factor in lowering SUA but not a necessary factor. Uric acid can function as an antioxidant, at low doses, but is toxic at high doses. Lowering SUA from 750 μM to 325 μM is beneficial.

### Example 6: Administering Molecular Hydrogen with Drugs that Lower Uric Acid

Drugs such as allopurinol, corticosteroids, and colchicine, used for treatment of high uric acid, can have serious side effects with long-term use. Administration of molecular hydrogen along with such drugs should lower the amount of drug needed to affect a desired reduction in serum uric acid. Side effects of these drugs should be reduced.

Also, co-administration of molecular hydrogen with one or more of these drugs should reduce pain since H₂ is known to reduce pain affected by inflammation. Uric acid crystals, in joints, is well known to cause severe pain.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

What is claimed is:

1. A method of accelerating production of nitric oxide (NO) by co-administering a generator of H₂ with a generator of nitric oxide (NO) as a formulation to a subject, wherein the generator of H₂ includes elemental magnesium metal powder and the generator of NO is a nitrate, and the formulation is free of amino acids.

2. The method according to claim 1, wherein the magnesium metal powder is in the formulation at a concentration of 0.01 to 50% w/w.

3. The method according to claim 1, wherein the generator of NO is present at a concentration of 0.01-50% w/w.

4. The method of claim 1, wherein the generator of NO is one or more of a salt of nitrate, a salt of nitrite, beet juice powder, celery powder, herbal powder, and vegetable powder containing nitrates.

5. The method of claim 4, further comprising ingesting a high fiber diet, which generates molecular hydrogen, before the co-administering.

6. The method of claim 5, wherein the high fiber diet contains fibers at a total content of 3-60 grams and wherein the fibers include one or more of beans, legumes, whole grains, cereals, vegetables, fruits, nuts, seeds, cellulose, hemicellulose, psyllium, resistant starch, inulin, wheat dextrin, oligosaccharides, pectin, glucomannan, xanthan gum, carrageenan, and combinations thereof.

7. The method of claim 6, wherein the high fiber diet is ingested 2-8 hours before ingestion of the generator of NO.

8. The method of claim 1, where the generator of H₂ further includes hydrogen-infused water.

9. The method of claim 1, where the generator of H₂ further includes H₂ gas which is administered by inhalation.

10. The method of claim 1, wherein the generator of H₂ further includes ascorbic acid and results in increases in salivary nitric oxide test strip measurements by up to 350%.

11. The method of claim 10, wherein ascorbic acid is present in the formulation at 0.001-75% w/w.

12. The method of claim 10, wherein the magnesium metal powder is present in the formulation at 0.01-50% w/w.

13. The method of claim 10, further comprising a modulator of eNOs (endothelial nitric oxide synthase) present in the formulation up to 25% w/w.

14. The method of claim 13, wherein the modulator of eNOS includes at least one of salts of nitrate, salts nitrites, acetyl salicylic acid, BH₄, folic acid, vitamin B₁, vitamin B₆, vitamin B₁₂, vitamin D₃, magnesium, and zinc.

15. The method of claim 1, wherein the co-administering of the generator of H₂ is sufficient to lower a blood level of uric acid.

16. The method of claim 15, wherein the salivary level of uric acid that is correlated to blood uric acid is determined.

17. The method of claim 1, wherein the formulation is in a form of capsules, tablets, powders, liquids, or hydrogels.

18. The method of claim 1, wherein the generator of H₂ is sufficient to reduce pain due to inflammation affected by uric acid.

19. The method of claim 18, wherein up to 50% ascorbic acid is added to the formulation to further reduce uric acid.

20. The method of claim 18, wherein the formulation further comprises one or more of allopurinol, corticosteroids, and colchicine.

21. The method of claim 18, wherein the subject is identified prior to the co-administering as having one or more diseases including cardiovascular disease, bone and joint deterioration, gouty arthritis, kidney disease, Alzheimer's disease, and vascular dementia.
